(19) United States
(12) Patent Application Publication
(10) Pub. No.: US 2026/0256070 A1
(43) Pub. Date: Sep. 3, 2026

(54) Tillandsia 'Amorosa'

(71) Applicant: Elly Bak, Rijsenhout (NL)

(72) Inventor: Elly Bak, Rijsenhout (NL)

(21) Appl. No.: 19/067,374

(22) Filed: Feb. 28, 2025

(51) Int. Cl. A01H 6/22; A01H 5/02

(52) U.S. Cl. CPC A01H 6/228; A01H 5/02

(57) ABSTRACT

The invention relates to the field of Vriesea and Tillandsia, specifically, the Vriesea x Tillandsia hybrid variety named ‘AMOROSA’. ‘AMOROSA’ is characterized by solid a growth habit; measuring about 25 cm in height with numerous, green color foliage (measuring about 40 cm length and about 4.5 cm in width), superior floral bract production; bracts are red in color (closest to RHS 46A), compound inflorescence, measuring about 65 cm in height and about 20 cm in diameter; and long-lasting habit. The present invention relates to methods using any plant parts or progeny of ‘AMOROSA’ for the purpose of reproducing ‘Amorosa’ or deriving additional new Vriesea x Tillandsia varieties. The present invention relates to seed, plants and plant parts produced by crossing ‘AMOROSA’ with any other plant. The present invention also relates to methods to produce new varieties of Vriesea x Tillandsia using the variety ‘AMOROSA’ and applying plant breeding techniques.

### FIELD OF THE INVENTION

The present invention relates to a new, distinct and stable hybrid of Vriesea x Tillandsia hybrid, hereinafter referred to as ‘AMOROSA’. The present invention relates to seeds which are the hybrid ‘AMOROSA’, as well as, plants and plant parts produced by these seeds which have all of the morphological and physiological characteristics of the hybrid ‘AMOROSA’. The present invention also relates to methods for producing these seeds and plants of the hybrid ‘AMOROSA’. Furthermore, the present invention relates to a method of producing progeny plants by crossing ‘AMOROSA’, as either the female or seed or male or pollen parent, with another Vriesea or Tillandsia plant and selecting progeny.

### BACKGROUND OF THE INVENTION

The present invention relates to a new, distinct and stable hybrid of Vriesea x Tillandsia hybrid, of the family Bromeliaceae, and hereinafter referred to by the variety denomination ‘AMOROSA’. The new hybrid ‘AMOROSA’ originated from a cross made in a controlled breeding program by the inventors in 2011, and then first flowered in 2014, in Assendelft, The Netherlands. The female or seed parent is an unnamed Vriesea inbred line identified by code 110052850249 (unpatented). The male or pollen parent is an unnamed Tillandsia selection identified by code 1100520001 (unpatented).

Vriesea and Tillandsia are members of the Bromeliaceae family. Vriesea and Tillandsia species are epiphytes. Vriesea and Tillandsia may be advantageously grown as pot plants for greenhouse or home use. Typically, the plants are shaded from direct sunlight. During the spring to autumn period, the central vase-like part of the leaf rosette is normally filled with water. Vriesea can be found growing in forested and rocky areas in Mexico, Central America, West Indies and South America. Tillandsia can be found growing in the deserts, forest and mountains of Central and South America, Mexico and southern United States.

Asexual propagation of Vriesea or Tillandsia is frequently performed by vegetative means through the use of tissue culture practices. Propagation of Vriesea and Tillandsia can also be from offshoots which can be detached from the mother plant and grown in a appropriate soil or bark mixture.

Methods for cultivation and crossing of Bromeliacaea are well known. For a detailed discussion, reference is made to the following publications, which are incorporated herein by reference: Benzing, David H., THE BIOLOGY OF THE BROMELIADS, Mad River Press, Inc. Eureka (1980); Zimmer, Karl, BROMELIEN, Verlag, Paul Parey, Berlin (1986); and Rauh, Werner, BROMELIEN, Verlag Eugen Ulmer, Stuttgart (1981).

A Vriesea inbred is produced by brother/sister crossing over several generations to produce a genetically homozygous plant selection. A hybrid cultivar is produced by crossing two genetically distinct inbred lines, collecting seeds produced by the cross, and germinating seeds so-produced to make hybrid plants. The hybrid seeds and plants produced by this method are uniform with respect their morphological and physiological characteristics.

The new ‘AMOROSA’ was developed through a controlled breeding program and exhibits unique, desirable and stable characteristics.

### SUMMARY OF THE INVENTION

The present invention provides Vriesea x Tillandsia plant selections that are solid, medium-sized, long-lasting hybrids with superior bract production and red inflorescence that exhibits good keeping quality. The present invention also provides Vriesea x Tillandsia plant selections with a compound inflorescence with a unique red color which distinguishes the new cultivar from typical hybrids.

These and other objectives have been achieved in accordance with the present invention which provides ‘AMOROSA’ as a new cultivar that is a product of a planned breeding program conducted by the inventor, Elly Bak, in Assendelft, The Netherlands. The female or seed parent is an unnamed Vriesea inbred line identified by code 110052850249 (unpatented). The male or pollen parent is an unnamed Tillandsia selection identified by code 1100520001 (unpatented).

Both parental cultivars have a sufficient degree of homozygosity such that the progeny of the cross are genetypically and phenotypically uniform. The new hybrid ‘AMOROSA’ therefore can be produced by sexual reproduction by crossing the parents identified by the codes 110052850249 and 1100520001 to produce a population of progeny plants, each of which has the combination of characteristics as herein disclosed for the new hybrid ‘AMOROSA’.

Seeds which are the hybrid ‘AMOROSA’ are produced by crossing the parents identified by the codes 110052850249 and 1100520001, and are deposited with the NCIMB Limited, Wellheads Place, Dyce, Aberdeen, AB21 7 GB, United Kingdom. 625 seeds were deposited with the NCIMB Limited on Jan. 27, 2025, with NCIMB accession number 44523.

### OBJECTS OF THE INVENTION

The present invention relates to a method of producing seed which are Vriesea x Tillandsia hybrid ‘AMOROSA’, by (a) crossing the unnamed Vriesea inbred line identified by code 110052850249 (unpatented) as the female or seed plant with the unnamed Tillandsia selection identified by code 1100520001 (unpatented) as the male or pollen parent, and the reciprocate cross with 1100520001 as the female parent and 110052850249 as the male parent, and (b) harvesting seeds produced from said crosses.

The present invention also relates to a method of producing plants having all the physiological and morphological characteristics of the Vriesea x Tillandsia hybrid ‘AMOROSA’ comprising the steps of (a) crossing of the unnamed Vriesea inbred line identified by 110052850249 (unpatented) as the female or seed parent with the unnamed Tillandsia selection identified by code 1100520001 (unpatented) as the male or pollen parent, and the reciprocate cross with 1100520001 as the female parent and 110052850249 as the male parent, (b) harvesting seeds produced from said cross; and (c) producing plants from said harvested seeds.

The present invention also relates to producing progeny plants from the cross of Vriesea x Tillandsia hybrid ‘AMOROSA’, as the female or male parent, with another Tillandsia or Vriesea plant, and selecting progeny plants from this cross.

### BRIEF DESCRIPTION OF THE PHOTOGRAPHS

The patent or application file contains at least one drawing executed in color. Copies of this patent or patent application publication with color drawings will be provided by the Office upon request and payment of the necessary fees.

The accompanying photographs illustrate the overall appearance of the new hybrid ‘AMOROSA’ showing the colors as true as is reasonably possible with colored reproductions of this type. Colors in the photographs may differ slightly from the color values cited in the detailed botanical description which accurately describes the color of ‘AMOROSA’.

FIG. 1 shows a side view perspective of the primary and top bracts produced by a typical potted, flowering plant of ‘AMOROSA’, at 16 months of age from potting size.

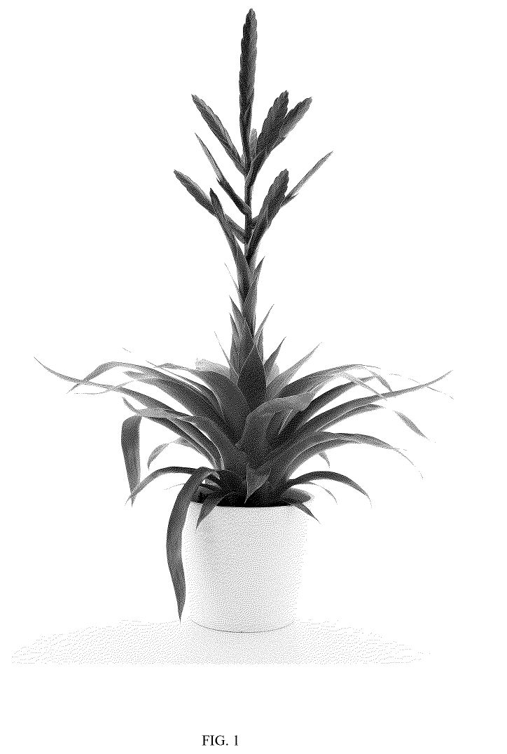

FIG. 2 shows a close-up top view perspective of the inflorescence and top bracts produced by a typical potted, flowering plant of ‘AMOROSA’, at 16 months of age from potting size.

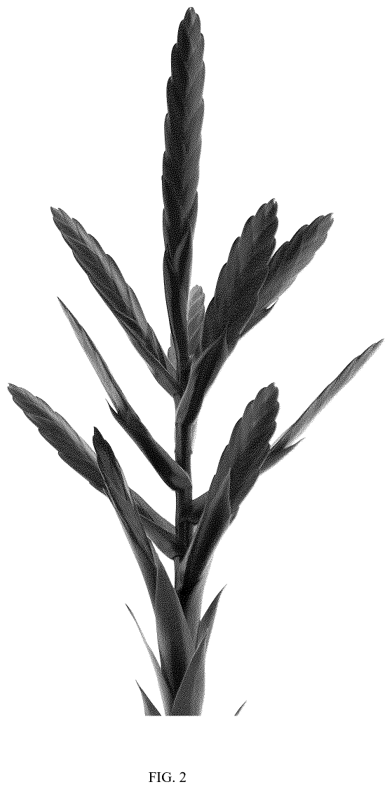

### DETAILED BOTANICAL DESCRIPTION

The present invention was created by the inventor, Elly Bak in 2011, and flowered for the first time in 2014 in Assendelft, The Netherlands.

This invention is directed to Vriesea x Tillandsia plant having all the morphological and physiological characteristics of the hybrid ‘AMOROSA’ produced from seeds which are the product of the cross of the unnamed Vriesea inbred line identified by code 110052850249 (unpatented) as the female or seed parent with the unnamed Tillandsia selection identified by code 1100520001 (unpatented) as the male or pollen parent. Both parents have a sufficient degree of homozygosity such that the progeny of the cross were, and continue to be, phenotypically uniform. The new hybrid ‘AMOROSA’ can therefore be produced by sexual reproduction by crossing of the selections identified by the codes 110052850249 and 1100520001 to produce a population of progeny plants, each of which has the combination of characteristics herein disclosed for the new hybrid ‘AMOROSA’.

The new hybrid ‘AMOROSA’ can also be produced by asexually reproducing progeny from the cross of the parental inbred lines identified by the codes 110052850249 and 1100520001. Asexual reproduction of the new cultivar by vegetative means by cuttings was first performed in 2015, in Assendelft, The Netherlands. The first ‘AMOROSA’ plants propagated through the use of such cuttings flowered in 2018, in Assendelft, The Netherlands, and have demonstrated that the new cultivar reproduces true-to-type and that the combination of characteristics as herein disclosed for the new cultivar are firmly fixed and retained through successive generations of asexual reproduction.

### BRIEF DESCRIPTION OF THE INVENTION

The following traits have been repeatedly observed and are determined to be unique characteristics of ‘AMOROSA’ which in combination distinguish this Vriesea x Tillandsia as a new and distinct cultivar:
1. Stemless growth habit;
2. Funnel-form rosette plant, measuring about 25 cm in height (above the pot when flowering);
3. Numerous, green color foliage (measuring about 40 cm in length and about 4.5 cm in width.
4. Superior floral bract production;
5. Bracts are red in color (closest to RHS 46A);
6. Compound inflorescence, measuring about 65 cm in height, when flowering and about 20 cm in diameter;
7. Long-lasting habit.

There are no known comparison cultivars.

‘AMOROSA’ has not been tested and observed under all possible environmental conditions. The phenotype of the new cultivar may vary with variations in environment such as temperature, light intensity, frequency of fertilization, composition of fertilizer, flowering treatment, day length and humidity, without any change in the genotype of the plant.

For example, substantial differences in plant height and diameter, number of leaves, can result depending on the size of the plant at the time that flowering is induced by flowering treatment. Since treatment to induce flowering disrupts normal watering and fertilization regimens. Flowering treatment of relatively smaller plants adversely affects the growth of the plant.

The aforementioned photographs, together with the following observations, measurements and values describe the new ‘AMOROSA’ as grown in a greenhouse in Assendelft, The Netherlands, under conditions which closely approximate those generally used in commercial practice. Plants of ‘AMOROSA’ were grown in a greenhouse with day temperatures ranging from 20° C. to 28° C. and night temperatures ranging from 18° C. to 23° C. No artificial lighting or photoperiodic treatments were conducted, but plants of ‘AMOROSA’ are forced into flowering.

Color references are made to the Royal Horticultural Society Colour Chart (RHS), 2001 edition, except where general colors of ordinary significance are used. Color values were taken under daylight conditions in a greenhouse in Assendelft, The Netherlands. The age of the plants of ‘AMOROSA’ described is about 17 weeks after flowering treatment.

### Classification

Botanical: Vriesea x Tillandsia

### Parentage

Female Parent: unnamed Vriesea inbred line identified by code 110052850249 (unpatented)
Male Parent: unnamed Tillandsia selection identified by code 1100520001 (unpatented)

### Plant

Height: About 25 cm (when flowering)
Width: About 60 cm
Shape: Funnel form rosette
Growth habit: Stemless
Plant Vigor: Good
Flowering Season: A fully grown plant can flower year round, starting 18 weeks after induction of natural light or through flowering treatment.
Cold Tolerance: Frost tender. Temperatures below 5° C. may damage plants.
Fragrance: None

### Foliage

Size of Leaf:
Length: About 40 cm (when flowering)
Width: About 4.5 cm
Overall Shape: linear lanceolate
Apex Shape: acuminate
Base Shape: Strap-like around central axis
Margin: Entire
Texture: Smooth
Orientation: Leaf blades arch continuously from base.
Color: Leaf color can vary somewhat depending on growing conditions.
Immature and Mature:
Upper surface: green, RHS 137A
Under surface: green, RHS 137A
Venation: None

### Inflorescence

Borne: Erect
Shape: compound
Size:
Length: About 65 cm in height when flowering
Diameter: About 20 cm
Individual flowers are fully enclosed by bracts and rarely visible.

### Bracts

Scape Bracts:
Quantity: About 18
arrangement: Alternate
Size:
Length: About 23.5 cm (lowest) to about 11.5 cm (scape bracts positioned just below the primary bracts).
Width: About 4.3 cm
Overall shape: Linear lanceolate
Apex shape: Acute
Base shape: fused
Margin: Entire
Texture: Smooth
Upper and under surfaces:
Scape bracts are greyed-purple, closest to RHS 184A with green, closest to RHS 137A just below the primary bracts

### Primary Bracts

Quantity: About 13 bracts
Arrangement: Alternate
Size: Length: About 9.5 cm
   Width: About 3 cm
Overall shape: linear lancelate
Apex shape: Acuminate
Base shape: Fused
Margin: Entire
Texture: Smooth
Color:
Upper and under surfaces: greyed-purple, closest to RHS 184A with green, closest to RHS 137A at the top.
Branches:
   Length: about 13 cm
   Width: about 3 cm
   Angle: about 45 degree
   Number: about 13
Floral bracts:
   Color: Red, closest to RHS 46A
   Length: about 5 cm
   Width: 2 cm
   Number: about 150

### Flower

General appearance: Flowers are fully enclosed by bracts and rarely visible. Each bract encloses one flower.
Quantity: about 150 flowers
Time of Bloom: A fully grown plant can bloom the whole year starting about 18-20 weeks after natural induction or through flowering treatment.
Duration of Bloom: Each flower can bloom one (1) day
Petals.
Number: 3 per flower
Length: About 4.5 cm
Width: About 0.6 cm
Overall Shape: Ligulate
Apex Shape: Obtuse
Base Shape: Fused
Color:
Upper and under surfaces: yellow
Sepals:
Number: 3 per flower
Length: About 3 cm
Width: About 0.7 cm
Overall Shape: Ligulate
Apex Shape: Acute
Base Shape: Fused
Color:
   Upper and under surfaces: yellow, closest to RHS 4A

### Reproductive Organs

Androecium:
Stamen:
   Number: 6 per flower
   Length: About 4.2 cm
   Diameter: About 0.1 mm
   Color: yellow, too small to distinguish RHS value
Anther:
   Length: About 0.6 cm
   Color: yellow, too small to distinguish RHS value
Pollen: not present
Gynoecium:
Pistil:
   Number: 1 per flower
   Length: About 3.7 cm
Stigma:
   Shape: 3-parted
   Width: About 0.3 cm
   Color: green, too small to distinguish RHS value
Style:
   Length: About 3.0 cm
   Color: yellow, too small to distinguish RHS value
Ovary:
   Position: Superior
   Shape: Conical
   Length: About 0.6 cm
   Diameter: About 0.3 cm
   Color: white
SEEDS/FRUIT: sterile hybrid, no seed or fruit produced.
DISEASE/PEST RESISTANCE: No observations made.
DISEASE/PEST SUSCEPTIBILITY: No observations made.

We claim:

1. Vrisea x Tillandsia ‘AMOROSA’, representative biological material deposited at NCIMB Limited, Wellheads Place, Dyce, Aberdeen, AB21 7 GB, United Kingdom and having accession number 44523.

2. A plant or a plant part thereof produced by growing the plant of claim 1, wherein the plant or plant part comprises at least one cell of Vrisea x Tillandsia ‘AMOROSA’.

3. A Vrisea x Tillandsia plant or part thereof, having the physiological and morphological characteristics of the plants of claim 1.

4. The tissue or cell culture of regenerable cells produced from the plant of claim 1.

5. The tissue or cell culture of claim 4, comprising tissues or cells from a plant part selected from the group consisting of leaves, vegetative cuttings, pollen, embryos, cotyledons, hypocotyl, meristematic cells, roots, root tips, pistils, anthers, flowers and stems.

6. A method of producing Vrisea x Tillandsia progeny comprising the steps of (a) crossing a plant of Vrisea x Tillandsia ‘AMOROSA’ as a female or male parent with another Vrisea x Tillandsia or Vrisea or Tillandsia plant or other plant and (b) selecting progeny.

7. The method according to claim 6, wherein the second plant is Vrisea x Tillandsia ‘AMOROSA’.

8. A Vrisea x Tillandsia seed that produces the Vrisea x Tillandsia plants of claim 1.

9. A method for developing a Vrisea x Tillandsia plant in a plant breeding program using plant breeding techniques, including crossing, recurrent selection, mutation breeding, wherein said mutation breeding selects for a mutation that is spontaneously or naturally induced or artificially induced, backcrossing, pedigree breeding, marker enhanced selection, haploid/double haploid production or transformation to a plant of Vrisea x Tillandsia ‘AMOROSA’, or it's parts, or progeny wherein application of said techniques results in development of a Vrisea x Tillandsia plant.
